(19) United States
(12) Patent Application Publication
(10) Pub. No.: US 2020/0206077 A1
(43) Pub. Date: Jul. 2, 2020

(54) AUTOLOGOUS STEM CELL THERAPIES FOR TREATMENT OF EYE DISEASE

(71) Applicant: AMD-OPTI, LLC, Willington, FL (US)

(72) Inventor: Maria Bartolomeo Grant, Franklin, IN (US)

(21) Appl. No.: 16/624,674

(22) PCT Filed: Jun. 26, 2018
(86) PCT No.: PCT/US2018/039413

§ 371 Date: Dec. 19, 2019

Related U.S. Application Data
(60) Provisional application No. 62/524,851, filed on Jun. 26, 2017.

(51) Int. Cl. A61H 39/00; A61K 35/15; A01N 1/02; A61K 9/00

(52) U.S. Cl. CPC A61H 39/002; A61K 35/15; A61P 27/02; A61K 9/0019; A01N 1/021

(57) ABSTRACT

Provided herein, in some embodiments, are methods that include electro stimulating at least one acupoint in a subject, isolating from a blood sample obtained from the subject cells that are immunocytochemically positive for CD 146 (CD146+), and administering to the subject a pharmaceutical composition of cells comprising non-cultured CD146+ stem cells. Also provided herein are pharmaceutical composition and kits comprising the non-cultured CD146+ stem cells.

### RELATED APPLICATION

This application claims the benefit under 35 U.S.C. § 119(e) of U.S. provisional application No. 62/524,851, filed Jun. 26, 2017, which is incorporated by reference herein in its entirety.

### BACKGROUND

Macular degeneration, also known as age-related macular degeneration (AMD or ARMD), is the leading cause of vision loss, affecting more than 10 million Americans—more than cataracts and glaucoma combined. Macular degeneration is caused by the deterioration of the central portion of the retina. Currently, macular degeneration is considered an incurable eye disease.

Diabetic retinopathy is another cause of vision loss, for which treatment options are limited. Diabetic retinopathy is a disease that can occur in patients with diabetes. High blood sugar levels cause damage to blood vessels in the retina. The blood vessels can swell and leak, or close, stopping blood from passing through, resulting in vision loss.

Diabetic macular ischemia (DMI) is also an important cause of visual loss in patients with diabetes. The abnormally high levels of blood sugar associated with diabetes can cause damage to the small blood vessels that supply oxygen and nutrients to the retina. Without the proper amount of oxygen and nutrients over time, the retina can become damaged (retinopathy).

There is a lack of effective therapies for these debilitating eye diseases.

Cellular therapies are potentially feasible therapies for various diseases, including some neurodegenerative diseases. Stem cells, for example, such as embryonic stem cells, induced pluripotent stem cells and mesenchymal stem cells, have the capacity to proliferate and differentiate into multiple cellular lineages. Stem cells isolated from a donor may be used in autologous manner to treat certain conditions/diseases. Hematopoietic stem cells, for example, are typically isolated from the peripheral blood of a donor, cultured, expanded ex vivo and re-introduced at the site of the disease of the donor.

### SUMMARY

Provided herein are autologous stem cell therapies for the treatment of eye diseases, such as macular degeneration, diabetic retinopathy and/or diabetic macular ischemia. The methods of the present disclosure advantageously do not require cell processing, such as culturing or cell expansion ex vivo, before re-introducing the donor stem cells back into the donor. Thus, the stem cells are not exposed to exogenous enzymes or growth factors, and are not cultured outside the body, which can increase exposure to contaminants that may adversely affect proliferative capacity of differentiation potential of the stem cells.

The methods of the present disclosure include the use of electroacupuncture to mobilize a highly therapeutically potent population of CD146⁺ stem cells (e.g., CD146⁺ mesenchymal stem cells) into peripheral blood from a variety of different organs. This isolated CD146⁺ stem cell population can be purified, concentrated and administered directly to a subject, without further ex vivo expansion. Using an exemplary method, as provided herein, a subject (e.g., a subject having an eye disease, such as macular degeneration) undergoes an electroacupuncture session, during which the LI-4, LI-11, GV-14, GV-20, ST-36 and/or LV-3 acupoints are stimulated. The subject then undergoes leukaphoresis, whereby white blood cells are separated from red bloods cells, and the red blood cells are returned to the subject. The white blood cells are then column purified using an anti-CD146 antibody. CD146⁺ stem cells are collected from the column, concentrated (e.g., in sterile saline) and directly administered to the subject. Unlike current autologous stem cell therapies, the entire procedure, from electroacupuncture through treatment, can be performed without ex vivo expansion within six hours.

Thus, in some aspects, the present disclosure provides methods that include electrostimulating at least one acupoint (acupuncture point) in a subject, and isolating from a blood sample (e.g., a peripheral blood sample) obtained from the subject mesenchymal stem cells that are immunocytochemically positive for CD146 (CD146⁺). In some embodiments, the methods further comprise administering to the subject a preparation of cells comprising non-cultured CD146⁺ mesenchymal stem cells. In some embodiments, the methods further comprise obtaining the blood sample from the subject, for example, through leukaphoresis.

Also provided herein are pharmaceutical compositions and kits comprising CD146⁺ mesenchymal stem cells produced by the electroacupuncture stem cell mobilization methods of the present disclosure.

### BRIEF DESCRIPTION OF THE DRAWINGS

FIG. 1: Representative diagram of the acupuncture points utilized in human and horse.

FIGS. 2A-2D: Electroacupuncture (EA) stimulation induced mesenchymal stem cell (MSC) mobilization. FIG. 2A: The percentage of human peripheral blood MSC increased in post EA-treatment (p=0.006, n=6). FIG. 2B: The percentage of circulating mesenchymal stem cells (MSCs) from adipose tissue (AD-MSC) are significantly elevated 2 h post EA-treatment; (p=0.033, n=4). FIGS. 2C, 2D. EA-mobilized MSC were expanded in vitro. After undergoing adipogenesis differentiation, EA-mobilized MSC developed fat deposits as seen by Oil Red staining (FIG. 2C), which were not seen in the undifferentiated control cells (FIG. 2D). Bars: 100 μm.

FIGS. 3A-3H: Rodent studies. EA increases sympathetic activation leading to browning of white adipose tissue (WAT) (increase in beige adipocytes). Anti-UCP1 immunofluorescence (red) detectable in inguinal subcutaneous adipose tissue (blue: adipocytes nuclei) from animals that underwent EA treatment (FIG. 3A) but not in control (FIG. 3B). Bars: 50 μm. The effects of EA can be duplicated by pharmacological disinhibition of hypothalamus with BMI: Lin-CD90hiCD44+ cells significantly (*p=0.027) increased in the plasma 4 h post injection (n=6) (FIG. 3C). EA-treated rodents exhibit reduced mechanical hyperalgesia (FIG. 3D), enhanced tissue remodeling (FIG. 3E) and increased serum IL-10 levels (FIG. 3F) following partial Achilles tendon rupture. *p<0.05, n=6-9. G, H. Small diameter primary afferent sensory neurons show sensitivity to pinch stimulus (FIG. 3G) and medium-large diameter neurons show sensitivity to EA (FIG. 3H) in Pirt-GCaMP3 mice with intact dorsal root ganglia (DRG) (arrows). p<0.0001, n=4.

FIGS. 4A-4F: EA-mediated sympathetic stimulation induces MSC release into the circulation in the horse. FIG. 4A: EA mobilized cells are highly proliferative and potentiate vasculogenesis, showing an increased colony-forming ability 2 h (p<0.05) post-EA at immune points (n=7), while cells obtained from the same horses with mock treatment or treatment at metabolic points did not. FIG. 4B: The EA-mobilized cells demonstrated high proliferative capacity, when plated in a single-cell assay, with over 50% proliferating into large colonies (p<0.001 vs. all groups). FIG. 4C: Equine and human peripheral blood mononuclear cells (PBMCs) were cultured to the third passage and then differentiated into key mesenchymal lineages: a, b—osteogenic potency, (Alizarin Red staining of calcium deposits); c, d—adipogenic response (Oil Red 0 staining of lipid deposits), weaker in horse (c) than in human (d) MSC. e-h: control conditions. i, j: chondrogenic differentiation (Alcian Blue staining of proteoglycans in the cell masses). Bars: 50 μm. FIG. 4D: In vivo angiogenesis assay of equine cells incorporated into a 3D type I pig skin collagen plug and placed subcutanously in NOD/SCID mice without (a) or together with human endothelial colony forming cells (hECFCs) (b, c). Bars: a, b: 50 μm; c: 10 μm. FIG. 4E: The hECFC-MSCs had a significant increase of arteriogenesis compared to hECFC alone (*p=0.02, n=5-7). FIG. 4F: After 48 h in vitro, cells were isolated, total mRNA was extracted and hey2 expression levels were quantified by qualitative reverse transcription polymerase chain reaction (qRT-PCR). Hey2 was elevated in the mixed cell treatment when compared to ECFC alone (p=0.006, n=4).

FIG. 5: EA-mobilized cells show a distinct origin from bone marrow-derived and adipose-derived equine mesenchymal stem cells. Principal component analysis of gene expression in equine EA-mobilized cells (EA-MSC), MSC from bone marrow origin (BM-MSC) and adipose-derived stem cells (ASC), as analyzed by GeneChip® microarrays.

FIG. 6 shows an example of a method of the present disclosure whereby a subject having macular degeneration undergoes an electroacupuncture session, white blood cells are later collected from the subject, the collected cells are passed over a column to isolate CD146⁺ stem cells, and the non-cultured CD146⁺ stem cells are concentrated and directly administered to the sub-Tenon's space of the subject's eye.

FIG. 7. Horse MSCs stained with CD90-PercpCy5.5 (human), CD146 APC (horse) and CD105 PE (horse).

FIG. 8: Schematic demonstrating the major findings of the Examples. EA administration promotes a localized signal to access the hypothalamus via the spinohypothalamic tract leading to stimulation of the hypothalamus and subsequent sympathetic signaling to the peripheral organs, inducing the browning of the white adipose tissue and the mobilization of mesenchymal-like cells from their tissue niches into the bloodstream. The addition of the nonselective beta blocker, propranolol, diminishes the benefit of EA treatment for both pain behavior and tendon repair.

### DETAILED DESCRIPTION

Electroacupuncture (EA) refers to the application of a pulsating electric current to a fine acupuncture needle(s) inserted in the skin as a means of stimulating pre-determined points throughout the body, referred to as acupoints (acupuncture points). Acupoints are located in areas of decreased electrical resistance and increased electrical conductivity in the body, attributed to both neural and vascular elements in the dermis or hypodermis. Animal studies show that electroacupuncture at particular acupoints results in the mobilization of mesenchymal stem cell populations into peripheral blood. Mesenchymal stem cells are multipotent stromal cells that can differentiate into a variety of cell types, including osteoblasts, chondrocytes, myocytes, and adipocytes

The use of EA at specific acupoints stimulates stem cell release into peripheral blood through the activation of the nervous system. Surprisingly, as provided herein, a particular therapeutically potent population of CD146⁺ mesenchymal stem cells can be harvested directly from the blood of EA-treated subjects and administered to the subjects without ex vivo expansion. Thus, the methods of the present disclosure provide low cost, low risk methods for autologous stem cell therapy.

Methods of the present disclosure include the step of electrostimulating at least one (e.g., at least 2, 3, 4, 5, 6 or more) acupoint in a subject. In some embodiments, the electrostimulating step comprises inserting acupuncture needles (e.g., one or two needles) into an acupoint and delivering electric impulses to the acupuncture needles. These acupoints receiving the electric impulses are considered electrostimulated.

The EA device used to deliver an electric current may deliver, for example, about 10-80 milliamps, or about 15-25 hertz (Hz), providing a voltage of about 40-80 volts (e.g., 40, 50, 60, 70 or 80 volts).

In some embodiments, the electrostimulating step comprises applying a (optionally pulsating) 10-70, 10-60, 10-50, 10-40, 10-30 or 10-20 milliamp electric current to acupuncture needle(s) inserted in at least one (e.g., at least 2, 3, 4, 5, 6 or more) acupoint in the subject. In some embodiments, the electrostimulating step comprises applying a (optionally pulsating) 10, 20, 30, 40, 50, 60, 70 or 80 milliamp electric current to acupuncture needle(s) inserted in at least one acupoint in the subject.

In some embodiments, the electrostimulating step comprises applying a (optionally pulsating) 15-20 or 20-25 Hz electric current to acupuncture needle(s) inserted in at least one (e.g., at least 2, 3, 4, 5, 6 or more) acupoint in the subject. In some embodiments, the electrostimulating step comprises applying a (optionally pulsating) 15, 20 or 25 Hz electric current to acupuncture needle(s) inserted in at least one acupoint in the subject. EA typically uses more than one needle at a time so that the impulses can pass from one needle to the other.

The duration of an electroacupuncture session may vary, although is rarely exceeds an hour. Thus, in some embodiments, the electrostimulating step comprises applying an electric current to at least one (e.g., at least 2, 3, 4, 5, 6 or more) acupoint for 10-60 minutes, or 15-45 minutes. In some embodiments, an electric current is applied to an acupoint for 10, 15, 20, 25, 30, 35, 40, 45, 50, 55 or 60 minutes. In some embodiments, an electric current is applied to an acupoint for 30 minutes. In some embodiments, an electric current is applied to an acupoint for 45 minutes.

Acupuncture needles are attached to a device that generates continuous electric pulses. Electroacupuncture stimulator devices are used to adjust the frequency and intensity of the impulse being delivered, depending on the condition being treated. Non-limiting examples of electroacupuncture stimulator devices that may be used as provided herein include the G6805 or G6805-2 electric stimulator. Other electroacupuncture devices, for example those designed for use in both electroacupuncture and transcutaneous electrical nerve stimulation (TENS) treatment, may be used as provided herein. The wave form used may be a continuous wave, a sparse and dense wave, or an intermittent wave.

Acupoints (also referred to as acupuncture points) are specific sites in the body for needle insertion in acupuncture and/or acupressure. Most acupoints are areas of high electrical conductance on the body surface. Acupoints are based on Traditional Chinese Medicine (TCM). More than four hundred acupoints have been described, with the majority located on one of the main meridians, pathways that run throughout the body and according to TCM transport life energy. TCM recognizes twenty meridians, cutaneous and subcutaneous in nature, which have branching sub-meridians believed to affect surrounding tissues. Twelve of these major meridians, commonly referred to as “the primary meridians,” are bilateral and are associated with internal organs. The remaining eight meridians are designated as “extraordinary,” and are also bilateral except for three, one that encircles the body near the waist, and two that run along the midline of the body.

When acupuncture was adopted in the U.S., a standard nomenclature was developed to unambiguously identify the acupoints on meridians. The World Health Organization (WHO) published A Proposed Standard International Acupuncture Nomenclature Report in 1991, listing 361 classical acupoints organized according to the fourteen meridians, eight extra meridians, 48 extra points, and scalp acupoints, and published Standard Acupuncture Nomenclature in 1993, focused on the 361 classical acupoints. Each acupoint is identified by the meridian on which it is located and its number in the point sequence on that channel.

The twelve primary meridians include lung (LU), large intestine (LI), stomach (ST), spleen (SP), heart (HT), small intestine (SI), bladder (BL), kidney (KI), pericardium (PC), triple energizer (TE), gallbladder (GB) and liver (LV). Thus, in some embodiments, at least one acupoint is thus selected from LU (1-12), LI (1-20), ST (1-45), SP (1-21), HT (1-9), SI (1-19), BL (1-67), KI (1-27), PC (1-9), TE, GB (1-44) and LV (1-14) acupoints. In some embodiments, at least one acupoint is selected from the group consisting of LI-4, LI-11, GV-14, and GV-20. In some embodiments, at least one acupoint is selected from the group consisting of LI-4, LI-11, GV-14, GV-20, ST-36 and LV-3. In some embodiments, at least one acupoint is LI-4. In some embodiments, at least one acupoint is LI-11. In some embodiments, at least one acupoint is GV-14. In some embodiments, at least one acupoint is GV-20. In some embodiments, at least one acupoint is ST-36. In some embodiments, at least one acupoint is LV-3.

In some embodiments, at least two (or two) acupoints are electrostimulated. For example: LI-4 and LI-11; LI-4 and GV-14; LI-4 and GV-20; LI-4 and ST-36; LI-4 and LV3; LI-11 and GV-14, LI-11 and GV-20, LI-11 and ST-36; LI-11 and LV-3, GV-14 and GV-20; GV-14 and ST-36; GV-14 and LV-3; GV-20 and ST-36; GV-20 and LV-3; or ST-36 and LV-3 may be electrostimulated. In some embodiments, at least one acupoint is located on the right side of the body and at least one acupoint is located on the left side of the body.

In some embodiments, at least three (or three) acupoints are electrostimulated. For example, at least three acupoints may comprise any combination of acupoints selected from LI-4, LI-11, GV-14, GV-20, ST-36 and LV-3.

In some embodiments, at least four (or four) acupoints are electrostimulated. For example, at least four acupoints may comprise any combination of acupoints selected from LI-4, LI-11, GV-14, GV-20, ST-36 and LV-3. In some embodiments, the at least four acupoints comprise LI-4, LI-11, GV-14 and GV-20.

In some embodiments, at least two (or at least three) sets of acupoints are electrostimulated. For example, the first set may include LI-4, LI-11, GV-14, and GV-20, and the second set may include GV-14, GV-20, ST-36 and LV-3.

Electroacupuncture as described herein is an application that causes mobilization of stem cells into the peripheral blood. It should be understood that the population of CD146⁺ stem cells isolated following electroacupuncture is not a naturally-occurring cell population.

In some embodiments, at least five (or five) acupoints are stimulated. In some embodiments, at least six (or six) acupoints are stimulated.

A subject may be a mammalian subject. For example, a mammalian subject may be human, simian, equine, bovine, porcine, ovine, caprine, canine, feline, or rodent. In some embodiments, the subject is a human subject. In some embodiments, the human subject has a neurodegenerative condition (e.g., result in progressive degeneration and/or death of nerve cells). In some embodiments, the human subject has an eye disease. Non-limiting examples of eye diseases include macular degeneration (age-related macular degeneration), diabetic macular ischemia, diabetic retinopathy, cataracts, glaucoma, and uveitis. Thus, the present disclosure provides methods of treating a human subject having an eye disease, such as macular degeneration (age-related macular degeneration), diabetic macular ischemia, diabetic retinopathy, cataracts, glaucoma, or uveitis. Such methods may comprise administering to a subject a pharmaceutical composition of cells comprising non-cultured CD146⁺ stem cells. In some embodiments, the methods comprise isolating from a blood sample obtained from the subject CD146⁺ stem cells and administering to the subject a pharmaceutical composition of cells comprising non-cultured CD146⁺ stem cells. In some embodiments, the methods comprise electrostimulating at least one acupoint in a subject, isolating from a blood sample obtained from the subject CD146⁺ stem cells, and administering to the subject a pharmaceutical composition of cells comprising non-cultured CD146⁺ stem cells.

A blood sample, in some embodiments, is a peripheral blood sample collected from the subject. Peripheral blood includes red blood cells (erythrocytes), white blood cells (leucocytes), and platelets, which are found within the circulating pool of blood and not sequestered within the lymphatic system, spleen, liver, or bone marrow. A blood sample may be collected simple by drawing blood using a needle in syringe, as is common practice in the art. Alternatively, a blood sample may be collected through leukaphoresis, as discussed below. Other blood collection methods may be used.

Following an electroacupuncture session (e.g., a 45 minute session), in some embodiments, a subject undergoes leukaphoresis (a specific type of apheresis) to separate white blood cells from other cellular components of the peripheral blood. The time between an electroacupuncture session and leukaphoresis may vary, although it is typically about 2 hours. In some embodiments, the time between an electroacupuncture session and leukaphoresis is 1-5 hours (e.g., 1, 2, 3, 4 or 5 hours). Leukaphoresis methods are known and generally include collecting blood through an intravenous tube in one arm, passing the blood through an apheresis machine that separates the white blood cells from the red blood cells, and returning the red blood cells through an intravenous tube in the other arm.

Immunocytochemically positive CD146 (CD146⁺) stem cells (e.g., CD146⁺ mesenchymal stem cells) are then isolated from the white blood cell population. CD146 (cluster of differentiation 146, also known as melanoma cell adhesion molecule (MCAM) or cell surface glycoprotein MUC18)) is a 113 kDa cell adhesion molecule used as a biomarker for mesenchymal stem cells isolated from multiple adult and fetal organs, and its expression may be linked to multipotency; mesenchymal stem cells with greater differentiation potential express higher levels of CD146 on the cell surface (Russell K C et al. Stem Cells 28 (4): 788-98, 2010). CD146⁺ mesenchymal stem cells typically do not express CD34 (are CD34⁻). CD146⁺ stem cells may be isolated for example by column purification or other purification method capable of separating CD146⁺ stem cells from a larger cell population. It should be understood that the term isolating, in the context of isolating CD146⁺ stem cells, refers to the process of removing CD146⁺ stem cells from neighboring non-CD146⁺ cell types. CD146⁺ stem cells that have been removed from a white blood cell population containing CD146⁻ cells are considered isolated CD146⁺ stem cells. The terms isolated and purified may be used interchangeably. A population, with respect to cells, refers to a group of cells that share at least one characteristic (e.g., immunocytochemically positive for CD146).

The present disclosure also provides methods for improving lameness and/or ultrasound appearance of naturally occurring acute superficial digital flexor tendon (SDFT) core lesions (tendonopathy), for example, in horses.

Following separation of the white blood cells from the red blood cells, CD146⁺ stem cells may be isolated by passing the white blood cell population through a purification column containing anti-CD146 antibodies (e.g., human monoclonal anti-CD146 antibodies, such as P1H12, HMB45 or OJ79 (EBIOSCIENCES™)). The anti-CD146 antibodies bind to CD146 on the surface of cells, thus the CD146⁺ stem cells are retained in the column. The CD146⁺ stem cells are then eluted from the column using an elution buffer, washed to remove the elution buffer, then concentrated, e.g., by centrifugation. The concentrated cells are then reconstituted to a concentration of 10,000-100,000 cells/μl (e.g., 1×10⁴, 1×10⁴, 3×10⁴, 4×10⁴, 5×10⁴, 6×10⁴, 7×10⁴, 8×10⁴, 9×10⁴, or 1×10⁵ cells/0) using a pharmaceutically acceptable buffer (e.g., sterile saline). In some embodiments, the concentrated cells are administered in a volume of 50-100 μl (e.g., 50, 60, 70, 80, 90 or 100 μl). Alternatively, CD146⁺ stem cells may be isolated by flow cytometry or other purification methods known in the art.

With current autologous stem cell therapies, the stem cells are isolated from a subject and then expanded ex vivo over the course of several days in a culture system. Such culture systems include stroma/stem cell co-culture, continuous perfusion and fed-batch cultures, and those supplemented with extrinsic ligands, membrane transportable transcription factors, complement components, protein modification enzymes, metabolites, or small molecule chemicals. Unlike current autologous stem cell therapies, the methods provided herein omit stem cell expansion ex vivo.

The CD146⁺ stem cells population (e.g., CD146⁺ mesenchymal stem cell population) used in the methods and pharmaceutical compositions of the present disclosure are not expanded ex vivo. Expansion ex vivo refers to the process of culturing a starting population of cells under conditions that permit cell division, thereby increasing the number of cells relative to the number of cells in the starting population. Thus, in some embodiments, the CD146⁺ stem cells are not expanded ex vivo prior to administering the cells to a subject. CD146⁺ stem cells that are not expanded in vivo are referred to herein as non-cultured CD146⁺ stem cells. In some embodiments, the non-cultured CD146⁺ stem cells are not exposed to (do not contact) culture/growth media (media that permits growth of cells). In some embodiments, the non-cultured CD146⁺ stem cells are not exposed to extrinsic ligands, membrane transportable transcription factors, complement components, protein modification enzymes, metabolites, and/or small molecule chemicals.

The methods and pharmaceutical compositions of CD146⁺ stem cells may be used to treat eye disease, for example. Non-limiting examples of such eye diseases include macular degeneration (age-related macular degeneration), diabetic macular ischemia, diabetic retinopathy, cataracts, glaucoma, and uveitis. Typically, a pharmaceutical composition of CD146⁺ stem cells is administered to one eye or both eyes of the subject. The cells may be administered by direct injection, for example, In some embodiments, the cells (e.g., a pharmaceutical compositions of CD146⁺ stem cells) are directly injected into the sub-Tenon's space of one eye or both eyes of a subject. In some embodiments, a pharmaceutical composition of CD146⁺ stem cells is administered systemically (e.g., intravenous injection or intrathecal injection).

One of the advantages of the methods as provided herein is that the time during which the CD146⁺ stem cells remain outside of the donor is minimized (e.g., because the stem cells are not expanded ex vivo). Thus, in some embodiments, the time between the step of isolating CD146⁺ stem cells from a population of white blood cells and administering to a subject a preparation of non-cultured CD146+ stem cells is less than an hour (e.g., less than 30 minutes). In some embodiments, the time between the step of isolating CD146⁺ stem cells from a population of white blood cells and administering to a subject a preparation of non-cultured CD146⁺ stem cells is 1-3 hours. In some embodiments, the time between the step of isolating CD146⁺ stem cells from a population of white blood cells and administering to a subject a preparation of non-cultured CD146+ stem cells is 1-6 hours.

Likewise, the entire process, from electroacupuncture through to administration of the pharmaceutical composition can be performed within 6 hours. In some embodiments, all steps of a method (the entire method) as provided herein are capable of completion within 6 hours, although longer time periods are contemplated. In some embodiments, all steps of a method (the entire method) as provided herein are capable of completion within 12 hours, within 18 hours, or within 24 hours.

Thus, a method of the present disclosure may comprise (a) electrostimulating at least one acupoint in a subject, (b) isolating from a blood sample obtained from the subject cells that are immunocytochemically positive for CD146 (CD146+), and (c) administering to the subject a pharmaceutical composition of cells comprising non-cultured CD146+ cells of step (b), all capable of being completed within a 6-hour, 12-hour, or 18-hour time period.

The phrase “the administering step (c) can be performed within X hours of step (b)” means that it is possible to proceed from step (b) to step (c) within X (e.g., 1, 2, 3, 4, 5, 6, 12, 18, or 24) of hours. It should be understood that in practice, proceeding from step (b) to step (c) may take longer than the stated number of hours if, for example, the medical professional performing step (c) is not able to proceed directly from step (b) to step (c) due to external circumstances, such as scheduling conflicts.

In some embodiments, the methods as provide herein may need to be repeated or performed on a regularly-scheduled basis. For example, a pharmaceutical composition of CD146⁺ stem cells may be administered to a subject (e.g., directly injected into the sub-Tenon's space of an eye) 3 months to 6 months (e.g., 3, 4, 5, or 6 months) following the initial treatment. In some embodiments, a pharmaceutical composition of CD146⁺ stem cells is administered to a subject 6-12 months (e.g., 6, 7, 8, 9, 10, 11, or 12 months) following initial treatment. In some embodiments, a pharmaceutical composition of CD146⁺ stem cells is administered to a subject every 3, 6, 9 or 12 months following initial treatment. In some embodiments, a pharmaceutical composition of CD146⁺ stem cells is administered to a subject weekly, bi-weekly, monthly, or bi-monthly. The actual amount (e.g., concentration and/or volume) administered, and rate and time-course of administration, will depend on the age, sex, weight, of the subject, the stage of the disease, and severity of disease being treated. Prescription of treatment, e.g., decisions on dosage is within the responsibility of general practitioners and other medical doctors.

Pharmaceutical compositions of CD146⁺ stem cells of the present disclosure may comprises 10,000 to 200,000 CD146⁺ stem cells. In some embodiments, a pharmaceutical composition comprises 10,000 to 100,000 CD146⁺ stem cells. For example, a pharmaceutical composition comprises 1×10⁴, 2×10⁴, 3×10⁴, 4×10⁴, 5×10⁴, 6×10⁴, 7×10⁴, 8×10⁴, 9×10⁴, 1×10⁵, or 1×10⁶CD146⁺ stem cells. In some embodiments, a pharmaceutical composition comprises 2×10⁴ to 1×10⁶, 3×10⁴ to 1×10⁶, 4×10⁴ to 1×10⁶, or 5×10⁴ to 1×10⁶CD146⁺ stem cells.

The majority of cells in a pharmaceutical composition of the present disclosure are CD146⁺ stem cells. Thus, in some embodiments, at least 60%, at least 70%, or at least 80% of the cells of a pharmaceutical composition are CD146⁺ stem cells. In some embodiments, at least 90% of the cells of a pharmaceutical composition are CD146⁺ stem cells. In some embodiments, at least 95% (e.g., 95%, 96%, 97%, 98%, 99% or 100%) of the cells of a pharmaceutical composition are CD146⁺ stem cells. In some embodiments, at least 98% of the cells of a pharmaceutical composition are CD146⁺ stem cells. In some embodiments, at least 99% of the cells of a pharmaceutical composition are CD146⁺ stem cells. In some embodiments, 90%-98%, 90%-99%, 95%-98%, or 95%-99% of the cells of a pharmaceutical composition are CD146⁺ stem cells.

In some embodiments, a pharmaceutical composition of CD146⁺ stem cells contains only CD146⁺ stem cells and sterile saline (e.g., phosphate buffered saline), although other carriers and/or excipients may be used.

While in many embodiments, CD146⁺ stem cells are collected and then directly administered to a subject, other embodiments are contemplated whereby some of the cells are banked (cryopreserved) for use in the subject (donor subject) for repeat administration. For example, a subject having macular degeneration may need at least two autologous CD146⁺ stem cell treatments, each separated by a 3-6 month time period. Rather than having the patient undergo electroacupuncture and leukaphoresis a second time, CD146⁺ stem cells collected from the subject during the first treatment session, may be cryopreserved/frozen, then later thawed and re-introduced into the subject during a second (and/or third and/or fourth, etc.) treatment session.

A pharmaceutical composition of CD146⁺ stem cells may be administered either alone or in combination with other treatments, either simultaneously or sequentially, depending on the condition to be treated.

Methods of preparing dosage forms of pharmaceutical composition are known, or will be apparent, to those skilled in the art. See Remington's Pharmaceutical Sciences, 20th Edition, 2000, pub. Lippincott, Williams & Wilkins. Where a pharmaceutical composition as provided herein is to be administered to an individual, administration is preferably in a prophylactically effective amount or a therapeutically effective amount, this being sufficient to benefit the subject.

The present disclosure further provides embodiments encompassed by the following numbered paragraphs:

1. A method, comprising:

(a) electrostimulating at least one acupoint in a subject;

(b) isolating from a blood sample obtained from the subject stem cells that are immunocytochemically positive for CD146 (CD146⁺); and

(c) administering to the subject a pharmaceutical composition of cells comprising non-cultured CD146⁺ stem cells of step (b).

2. The method of paragraph 1, wherein step (a) comprises electrostimulating at least two acupoint in the subject.
3. The method of paragraph 1 or 2, wherein the at least one acupoint is selected from the group consisting of LI-4, LI-11, GV-14, GV-20, ST-36 and LV-3.
4. The method of any one of paragraphs 1-3, wherein step (a) comprises applying a pulsating 15-25 hertz (Hz) electric current to acupuncture needle(s) inserted in the at least one acupoint in the subject.
5. The method of paragraph 4, wherein the electric current is applied for at least 15 minutes.
6. The method of paragraph 5, wherein the electric current is applied for 15-60 minutes.
7. The method of paragraph 6, wherein the electric current is applied for 45 minutes.
8. The method of any one of paragraphs 1-7, wherein step (b) comprises subjecting the subject to leukaphoresis to separate white blood cells from red blood cells.
9. The method of paragraph 8 further comprising passing the white blood cells through purification column containing anti-CD146 antibodies.
10. The method of paragraph 9 further comprising eluting CD146⁺ stem cells bound to the anti-CD146 antibodies in elution buffer.
11. The method of paragraph 10 further comprising washing the eluted CD146⁺ stem cells to remove the elution buffer.
12. The method of paragraph 11 further comprising concentrating the CD146⁺ stem cells in sterile saline to produce the pharmaceutical composition of CD146⁺ stem cells.
13. The method of paragraph 12, wherein the pharmaceutical composition comprises 10,000 to 100,000 CD146⁺ stem cells.
14. The method of paragraph 12 or 13, wherein at least 90% of the cells of the pharmaceutical composition are CD146⁺ stem cells.
15. The method of paragraph 14, wherein at least 95% of the cells of the pharmaceutical composition are CD146⁺ stem cells.
16. The method of paragraph 15, wherein at least 98% of the cells of the pharmaceutical composition are CD146⁺ stem cells.
17. The method of any one of paragraphs 1-9, wherein the administering step (c) is performed within an hour of step (b).
18. The method of paragraph 17, wherein the administering step (c) is performed within three hours of step (b).
19. The method of any one of paragraphs 1-18, wherein steps (a)-(c) are completed within 6 hours.
20. The method of paragraph 19, wherein steps (a)-(c) are completed within 18 hours.
21. The method of any one of paragraphs 1-20, wherein the subject is a mammalian subject.
22. The method of paragraph 21, wherein the mammalian subject is a human subject.
23. The method of paragraph 22, wherein the human subject has an eye disease.
24. The method of paragraph 23, wherein the eye disease is selected from macular degeneration, diabetic macular ischemia, and diabetic retinopathy.
25. The method of paragraph 24, wherein step (c) comprises injecting the pharmaceutical composition in at least one eye of the subject.
26. The method of paragraph 25, wherein step (c) comprises injecting the pharmaceutical composition in both eyes of the subject.
27. The method of paragraph 25 or 26, wherein step (c) comprises injecting the pharmaceutical composition in the sub-Tenon's space of one or both eyes of the subject.
28. The method of any one of paragraphs 1-27 further comprising repeating steps (a)-(c) at least once following a 3- to 6-month time period.
29. A method, comprising:

(a) electrostimulating at least two acupoints in a human subject having macular degeneration;

(b) isolating from a blood sample obtained from the subject stem cells that are immunocytochemically positive for CD146 (CD146⁺); and

(c) directly injecting into the sub-Tenon's space of at least one eye of the subject a pharmaceutical composition of cells comprising non-cultured CD146⁺ stem cells of step (b).

30. The method of paragraph 29, wherein the at least two acupoints are selected from the group consisting of LI-4, LI-11, GV-14, GV-20, ST-36 and LV-3.
31. A method, comprising:

(a) electrostimulating at least one acupoint in a subject; and

(b) isolating from a blood sample obtained from the subject stem cells that are immunocytochemically positive for CD146 (CD146⁺).

32. The method of paragraph 31 further comprising cryopreserving non-cultured CD146⁺ stem cells.
33. The method of paragraph 32 further comprising thawing the cryopreserved non-cultured CD146⁺ stem cells, and administering the non-cultured CD146⁺ stem cells to the subject.
34. A method, comprising:

directly injecting into at least one eye of the subject having an eye disease a pharmaceutical composition of cells comprising non-cultured cells that are immunocytochemically positive for CD146 (CD146⁺) stem cells.

35. The method of paragraph 34, wherein the pharmaceutical composition of cells is directly injected into the sub-Tenon's space of at least one eye of the subject.
36. A method, comprising administering to a subject having an eye disease a pharmaceutical composition comprising non-cultured cells that are immunocytochemically positive for CD146 (CD146⁺), wherein the non-cultured CD146⁺ stem cells are isolated from a subject subjected to electroacupuncture, wherein the preparation optionally comprises a pharmaceutically acceptable carrier and/or excipient.
37. The method of paragraph 36, wherein the eye disease is selected from macular degeneration, diabetic macular ischemia, and diabetic retinopathy
38. The method of paragraph 37, wherein the eye disease is macular degeneration.
39. A pharmaceutical composition comprising non-cultured cells that are immunocytochemically positive for CD146 (CD146⁺), wherein the non-cultured CD146⁺ stem cells are isolated from a subject subjected to electroacupuncture, wherein the preparation optionally comprises a pharmaceutically acceptable carrier and/or excipient.
40. The pharmaceutical composition of paragraph 39, wherein the pharmaceutically acceptable carrier and/or excipient is sterile saline.

### Examples

### Example 1. Role of the Hypothalamus in the Electroacupuncture Response

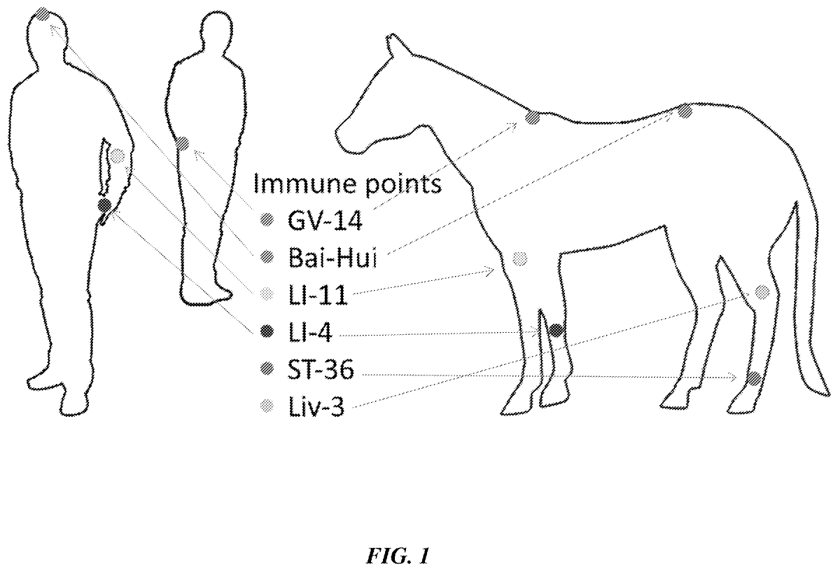

While both anatomical characteristics and local mediators of signaling are associated with specific acupoints, the mechanism responsible for the beneficial systemic effects and healing associated with acupuncture still lacks understanding. We have shown that electroacupuncture (EA), using a set of points (1: LI-4, LI-11, Du-14 and Du-20; FIG. 1) that we determined, was associated with mobilization of MSC. The systemic beneficial effects of EA may be centrally-driven, therefore we sought to determine the relationship between the hypothalamus and other brain structures, which is termed “connectivity”. The hypothalamus plays a critical role as a primary homeostatic center in the brain and contains neurons with important projections to other limbic sites and sympathetic nuclei directly communicating with the periphery. To determine whether the hypothalamus was involved in the EA response, arterial spin labeling fMRI was performed in healthy human subjects (n=6) during a single EA session. Acupoints LI-4 and LI-11 were stimulated for a total of 16 minutes (FIG. 1). Data were collected using a whole body Philips Achieva 3T scanner and a 32-channel head coil (Invivo Corporation, Gainesville, Fla., USA). Connectivity was derived from 4 time points: baseline, 0-8 minutes during EA, 8-16 minutes during EA and immediately post-EA. EA-stimulation produced changes in humans in the strength of functional connectivity within the hypothalamus and between the hypothalamus and adjacent brain regions compared to baseline and the post-EA period (data not shown). These results were also observed in male Sprague-Dawley rats by BOLD fMRI, suggesting that rats and humans exhibited a similar response in terms of observed connectivity (data not shown).

### Example 2. Characterization of Human Electroacupuncture-Mobilized Mesenchymal Stem Cells as Adipocyte-Derived Mesenchymal Stem Cells

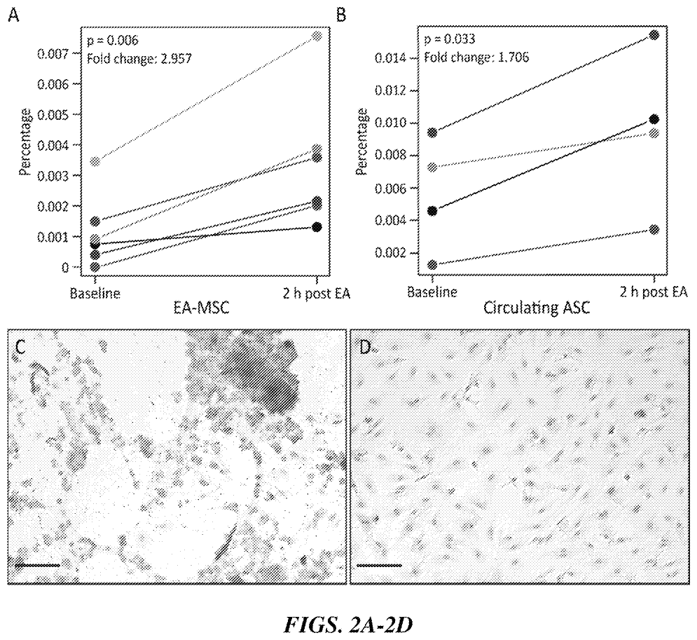

We next examined the populations of cells mobilized by EA in the human subjects that underwent fMRI in Example 1. Due to limitations regarding the fMRI equipment used, only points Du-14, LI-11 and LI-14 were able to be used in this experiment. We observed an increase in MSC in the peripheral blood two hours following EA in all subjects (FIG. 2A) while total lymphocyte numbers did not change. To further characterize the MSC population and potentially determine whether they were released from adipose stores, the levels of CD34+CD45−CD31− cells were examined. Two hours following completion of EA, CD34+CD45−CD31− cells were increased in 4 of 6 individuals (FIG. 2B) and the response was proportional to the body mass index of the subjects as previously noted. Two individuals with body mass index less than 18.5 did not demonstrate an increase in this population. Human EA mobilized cells MSC were expanded in vitro and underwent adipogenic differentiation. Adipogenesis potential was confirmed by the cells' ability to form lipid droplets as established through Oil Red 0 staining (FIG. 2C, 2D). Since hypothalamic activation preceded the mobilization of circulating MSC, we propose that the EA-induced connectivity changes contributed to the subsequent release of these cells into the peripheral blood. Exogenous administration of epinephrine or dopamine in rats resulted in a similar increase of Lin-CD90HICD44+ cell population (rat MSCs) into the circulation, supporting the role of CNS in mobilization of MSC and in EA activating these key CNS centers.

### Example 3. Small Rodent Studies

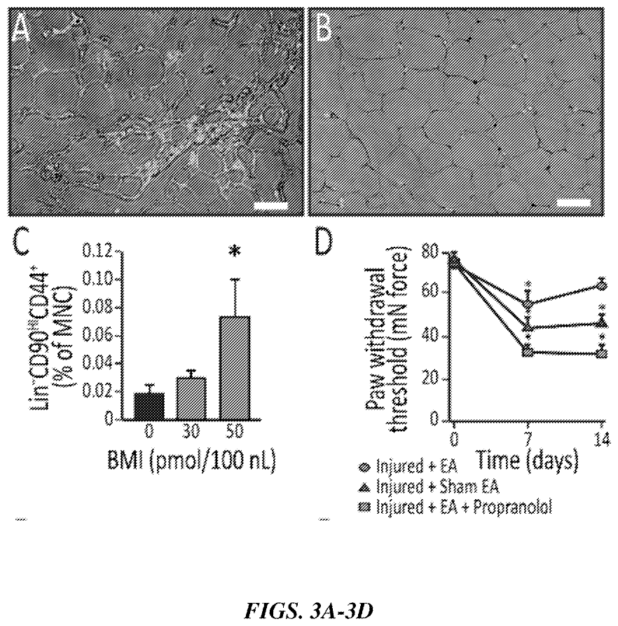

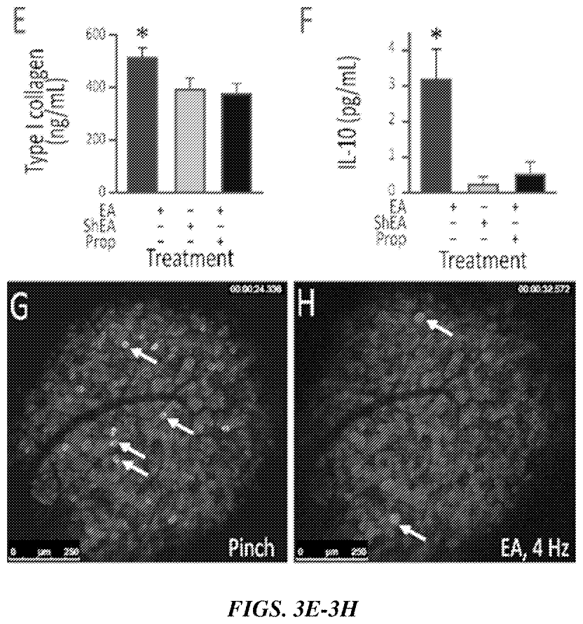

To further understand the mechanism(s) of EA effects, we performed a series of experiments on small rodents, as follows. To confirm that EA can activate the autonomic nervous system, ANS, we asked whether EA was associated with browning of WAT. The function of brown and beige adipocytes is muscle-independent thermogenic energy dissipation, which relies on the function of uncoupling protein 1 (UCP1). Following 14 days of EA (every other day) in rats, UCP1 immunofluorescence of inguinal subcutaneous WAT was increased, while no changes were detected in intraperitoneal WAT (FIG. 3A-3B).

To confirm that activation of the ANS can mediate release of MSC, we performed stereotaxic injections with the GABAA receptor antagonist bicuculline methiodide (BMI, 50 pmol) in the dorsomedial regions of the tuberal hypothalamus of rats, to disinhibit these regions. BMI increased the percentage of circulating Lin-CD90+CD44HI cells (p=0.027) at 4 h post injection of BMI (FIG. 3C) in the absence of EA.

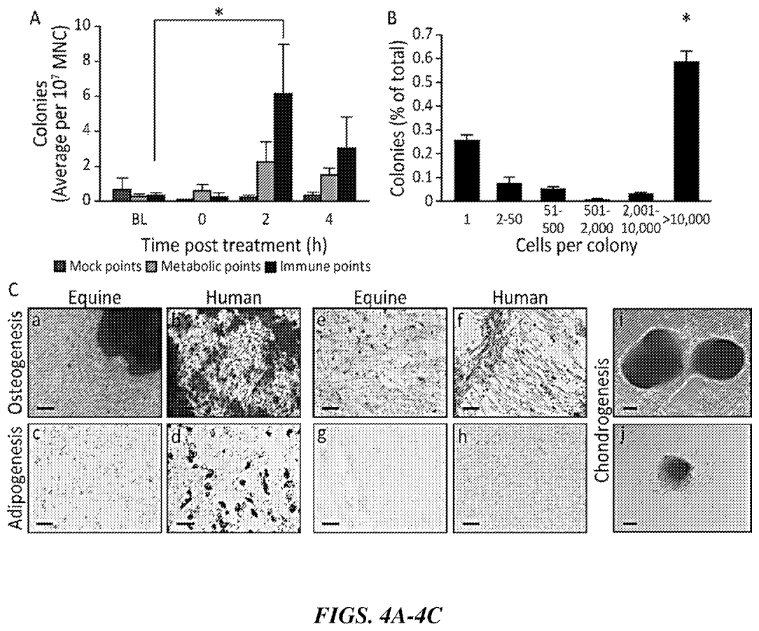

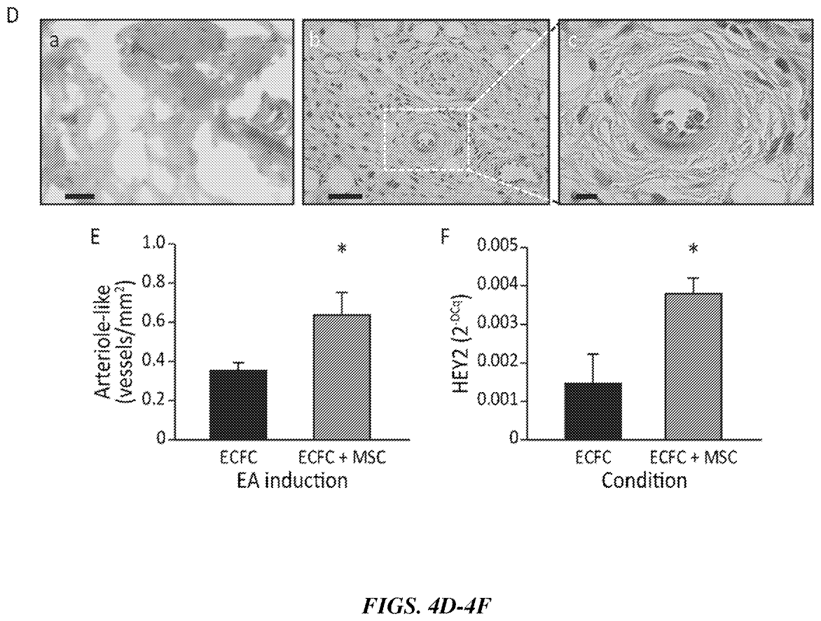

To address the possible anti-inflammatory and analgesic effects of EA-mobilization of circulating MSC, we analyzed the contribution of EA treatment on injury-induced hyperalgesia in rats in the presence and absence of the β-blocker, propranolol. Treatments administered every other day for two weeks following injury included EA (applied to immune acupoints LI-4, LI-11, Du-14 and Du-20), sham EA (applied to skin not associated with an acupoint) or EA plus propranolol (a potential inhibitor for sympathetic effects of EA). Mechanical hyperalgesia (assessed at both day 7 and 14) was considerably decreased (i.e., able to tolerate more pressure) in injured rodents subjected to EA at immune acupoints (FIG. 3D). Type I collagen, which is synthetized by fibroblasts at later stages of healing, was also significantly enhanced by EA (FIG. 4E). At the same time, injured rodents treated with EA significantly increased the endogenous anti-inflammatory cytokine IL-10 in plasma (FIG. 3F).

To distinguish the afferent stimulation of the hypothalamus by peripheral acupoints, we treated Pirt-GCaMP3 mice with either a noxious hind paw pinch (100-gram force) or EA directed at the hind limb acupoints. Hind paw stimulation evoked robust and transient Ca2+increases in 12.7±3.0 neurons per DRG (FIG. 3G) of which nearly all were small diameter neurons (<20 μm) suggestive of pain fibers, while placement of acupuncture needles alone did not elicit activity. Following EA stimulation; many activated neurons were medium (20-25 μm) to large (>25 μm) diameter, with an average of 6.1±4.0 neurons per ganglia; suggesting activation of touch fibers (FIG. 3H).

Thus, we confirmed that the effect of EA we observed is uniform across three species, and horse studies further validated the findings in the fourth species (vide infra).

### Example 4. Electroacupuncture Studies on Horses

We examined the immune points in horses undergoing treatment for different conditions (n=30), and as control for the specificity of these immune points, horses received EA at BL-20, SP-6 and ST-36 (FIG. 1) (metabolic points, n=4) [16], and at sham points (i.e., not used in acupuncture, n=4), or no intervention (n=4). A fine needle (0.30 mm×75 mm, Suzhou Medical Instrument Factory, Jiangsu, China) was inserted into each acupoint. Six acupoints (3 sets) were specifically used, Du-14 with Du-20, the left LI-4 with the left LI-11, and the right LI-4 with the right LI-11. Each set was electrically stimulated with 20 Hz for 45 minutes using a JM-2A EA Instrument (Wuxi Jiajian Medical Instrument, Inc., Wuxi, China). For the metabolic point group, a similar experimental paradigm was utilized but the six acupoints (3 sets) were the left BL-20 with the right BL-20, the right ST-36 with the left SP-6, the right ST-36 with the right SP-6. For the sham control group, six non-acupuncture points (3 sets) were used, the left BL-20 with the spot 1 cm lateral to Du-14 with the spot 1 cm lateral to Du-20, the spot 1 cm lateral to the left LI-4 with the spot 1 cm lateral to the left LI-11, the spot 1 cm lateral to the right LI-4 with the spot 1 cm lateral to the right LI-11. Blood (30 mL/time point) was collected before EA (0 h) and every two hours after the treatment for 6 hours (2 h, 4 h, 6 h). To avoid an artifact due to intrinsic circadian rhythmicity of stem cell release, all procedures were done starting at 9 in the morning, and blood was collected at the same times for all animals.

Mononuclear cells were isolated using Ficoll-paque (GE Healthcare Bio-sciences, Pittsburgh, Pa.) density gradient separation and centrifuged at room temperature at 1100×g for 30 minutes. Cell pellets were resuspended in a buffer consisting of PBS containing 2% fetal bovine serum (Thermo Scientific, Waltham, Mass., USA). A red blood cell lysis step was included by adding 2 ml use of ammonium chloride solution (Stem Cell Technologies, Vancouver, Canada) and incubating the mixture for 15 minutes at 4° C. Cells were then washed in the buffer twice and centrifuged at room temperature at 300×g for 5 minutes.

Due to the limited availability of equine MSC antibodies compared to humans and mice, stem cell mobilization into peripheral blood was confirmed by measuring circulating cell colony-forming ability in vitro. While colony-forming cells were rarely seen at baseline, colony-forming ability was easily detected in blood samples obtained 2 and 4 h after EA (FIG. 4A), the identical time points examined in humans and rats. Blood collected at 2 and 4 h post EA using the mock acupoints in the same horses did not give rise to colonies in vitro. Importantly, and representing a more critical control than simply sham acupoints, the use of metabolic points similarly did not give rise to significantly more colonies in vitro (FIG. 4A). To further verify the stem/progenitor characteristics of the equine cells, clonogenic potential was determined using single cell assays. EA-mobilized cells showed robust clonogenic potential, with over 75% proliferating into 2 or more cells, and over 50% of them resulting in large colonies of ≥10,000 cells, levels of proliferation that are generally reflective of stem/progenitor cells (FIG. 4B). The MSC origin of the mobilized colony forming cells was confirmed by them in vitro differentiation into osteocytes as demonstrated by positive staining for calcium deposits, (FIG. 4C panels a, b (controls: panels e, f), as well as their adipogenic differentiation (FIG. 4C panels c, d (controls: panels g, h) and chondrogenic differentiation (FIG. 4C, panels i, j) supporting that equine EA-mobilized cells display MSC lineage characteristics. When the EA-mobilized equine cells were examined in the in vivo angiogenesis assay, the cells did not directly form blood vessels (FIG. 4D, panel a) or lumenize; thus supporting a non-endothelial origin. However co-implantation of the EA-mobilized equine cells with human cord blood derived endothelial colony forming cells (ECFCs) significantly enhanced ECFC vasculo-genesis (FIG. 4D, panels b, c) and the number of human vessels with an arterial morphology (FIG. 4E). However, when the EA-mobilized MSC were co-cultured with hECFC in vitro, a significant increase in hey2 expression was observed in the endothelial cells, which indicated that the addition of the equine cells promoted arteriogenesis, as the Notch signaling pathway is known to be active in arterial vascular endothelial cells (FIG. 4F). Overall, these data support that EA-mobilized cells display MSC characteristics and enhance human ECFC vasculogenesis and arteriogenesis.

### Example 5. Gene Array Studies

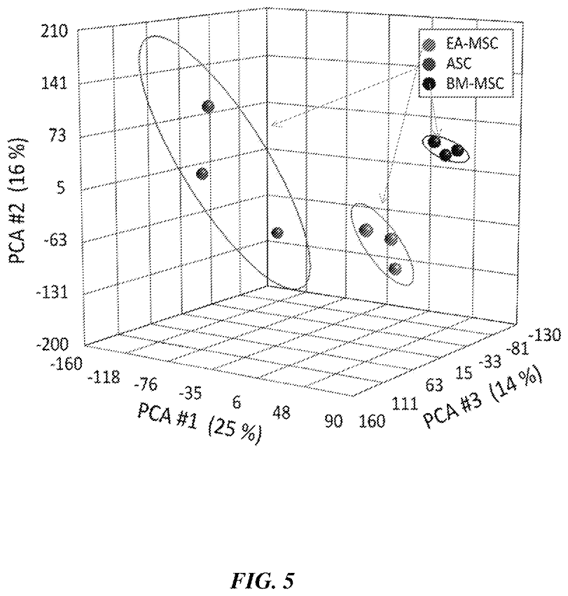

To compare the EA-mobilized MSC (EA-MSC) to other MSC populations derived from either depots of adipose tissue (ASC) or the bone marrow (BM-MSC), we performed gene array studies. Of the ˜30,000 genes present on the EquGene-1_0-st GeneChip®, 678 showed significant differences between EA-MSC and BM-MSC, 1164 between the EA-MSC and ASC and 1193 between ASC and BM-MSC (all p<0.05 and absolute fold change >2). Principal component analysis mapping (FIG. 5), hierarchical clustering and partitioning clustering showed that the EA-MSC, BM-MSC and the ASC segregated into distinct groups. This suggests either that the EA-mobilized MSC population may be derived from a source distinct from either adipose tissue or bone marrow, or that their mobilization into the systemic circulation modified their gene expression from that of the BM-MSC or ASC obtained directly from their tissue source.

### Example 6. Transcutaneous Electrical Stimulation

Contemporary interpretation of EA must consider contributions from the dry needle as well as the electrical stimulation itself, as performed in clinical applications such as transcutaneous electrical stimulation (TENS). Therefore, we next asked whether TENS performed with adhesive electrodes versus EA placed at immune acupoints LI-4, LI-11 and GV-14 and GV-20 would result in similar MSC mobilization. All horses (n=6) were stimulated with TENS using an EMPI Select TENS unit (EMPI Inc., St. Paul, Minn.) with the same EA electrical parameters of 20 Hz, intensity to palpable “tapping” (commonly used 1-4 mA) for 45 minutes at LI-4, LI-11 and GV-14 and GV-20. This resulted in mobilization of MSC as confirmed by growth of MSC cultures (data not shown) from the 2 h peripheral blood sample obtained following TENS.

Thus, the above studies support that EA activates the hypothalamus leading to mobilization of MSC, which can be ex vivo expanded while preserving the phenotypic and functional characteristics of MSC. Similar responses to those observed with forelimb immune points were observed with injections of either epinephrine or dopamine and with pharmacological disinhibition of the tuberal hypothalamus with BMI. Furthermore, these electrical activities could be replaced by TENS stimulation at the precise EA points. Thus, the mobilization of MSCs is likely centrally-mediated through hypothalamic activation and subsequent ANS activity. There has been increasing interest and evidence supporting the role of the ANS as a regulator of immune cell release, as appropriate activation and signaling is necessary to mobilize these cells into the blood stream. EA-induced mobilization of circulating MSC may directly or indirectly modulate anti-inflammatory and immunomodulatory properties in vivo, as we show by the increase in serum IL-10 levels and the observed reduced mechanical hyperalgesia. This would suggest that EA limits the production of nociceptive pro-inflammatory cytokines and serve to enhance tissue remodeling following tendon injury.

### Example 7. Ex-Vivo Expansion and IV Infusion of EA-MSC into Horses

We used the information thus obtained in the four species under study to further the application of EA-stimulated MSC release into the peripheral circulation to provide pain relief, immune modulation, and tissue regeneration.

The purpose of this work was to determine if EA-mobilized peripheral blood-derived MSC are effective for the treatment of lameness in horses. All experiments were conducted in accordance with guidelines of protocol 10904 of the Indiana University-Purdue University at Indianapolis IACUC.

Horse blood (70 mL) was collected 2 h after EA treatment and mononuclear cells were isolated by gradient centrifugation. Blood was diluted with 1 volume PBS supplemented with 2% fetal bovine serum (Thermo Scientific, Waltham, Mass., USA), and layered on one volume of Ficoll-Paque (GE Healthcare Bio-sciences, Pittsburgh, Pa.) in 50 mL conical tubes. After 30 min centrifugation at ˜900×g, accel 5, brake 1, room temperature, the buffy coat containing mononuclear cells was collected, and transferred to a new tube, filled with 2% FBS in PBS, supplemented with 1 mM EDTA, and centrifuged 10 min, ˜400×g, accel 9, brake 9, room temperature. A red blood cell lysis step was performed by adding over the pellet 2 mL of ammonium chloride solution (Stem Cell Technologies, Vancouver, Canada) and incubating the mixture for 15 minutes at 4° C. Cells were then washed two more times.

For in vitro expansion, isolated cells were plated on cell culture grade plastic 6-well plates at 10⁷ cells/well. Three mL of medium (1:1 dilution of aMEM (Lonza, Walkersville, Md., USA) and EBM-2 (Lonza), with 15% FBS and 1% antibiotics; the EBM-2 was supplemented with two SingleQuots™ Kits per one Liter). Cells were allowed to attach for 4 days and medium was changed every 2-3 days. Colonies usually appeared 14-21 days after plating, and reached 70-80% confluency at 21-30 days post-plating. After the MSC were expanding, the cultures were switch to equine serum (15%) for 3 passages and then the last two passages the cells are grown in the horses own serum.

The equine MSCs were successfully frozen. Cells were passaged when cultures reached 80% confluence by detaching with 0.25% Trypsin-EDTA solution (Thermo Scientific), washed twice in cell culture medium and filtered through sterile 40 μm cell strainers (BD Falcon™, BD Biosciences, San Jose, Calif.). When 50 million cells were obtained they were frozen by resuspending 5×10⁶ cells/mL in a mixture of cell culture medium supplemented with 30% equine serum and 10% dimethyl sulfoxide (DMSO, Sigma Aldrich, Carlsbad, Calif.) and slowly cooling to −80° C. Once frozen, they were then moved to liquid nitrogen for cryopreservation until infusion.

For the 24 horse study that provides the preliminary data for the current FDA submission, autologous frozen MSC were frozen in liquid nitrogen and then maintained on dry ice until the time of administration. The cells were quickly thawed in a water bath at 37° C. and immediately drawn into a sterile syringe and suspended to 30 mL with sterile Calcium-Magnesium free Phosphate buffered saline (Thermo Scientific). The mix was infused systemically into the jugular vein slowly over 3-5 minutes. Horses were monitored immediately following infusion to detect any adverse effects, and then kept under observation for 6 weeks to determine if there was any change in health. The American Association of Equine Practitioners (AAEP)'s lameness scale was used as a standard for grading improvement in lameness injuries in the animals.

A total of 24 horses with osteoarthritis were treated in the initial study.

TABLE 1
Lameness scored according to AAEP

| Horse | Age | Gender | Initial | Final |
|---|---|---|---|---|
| 1 | 27 | G | 3 | 1 |
| 2 | 17 | G | 2 | 0 |
| 3 | 9 | F | 3 | 0 |
| 4 | 15 | F | 4 | 2 |
| 5 | 35 | G | 3 | 0 |
| 6 | 9 | G | 3 | 0 |
| 7 | 15 | G | 3 | 0 |
| 8 | 5 | G | 2 | 0 |
| 9 | 20 | F | 4 | 2 |
| 10 | 14 | F | 1 | 1 |
| 11 | 17 | F | 1 | 1 |
| 12 | 13 | F | 1 | 1 |
| 13 | 7 | G | 2 | 1 |
| 14 | 6 | M | 2 | 0 |
| 15 | 7 | M | 2 | 1 |
| 16 | 7 | G | 1 | 0 |
| 17 | 9 | G | 1 | 0 |
| 18 | 11 | G | 2 | 1 |
| 19 | 16 | G | 1 | 0 |
| 20 | 6 | F | 1 | 0 |
| 21 | 8 | G | 2 | 1 |
| 22 | 10 | G | 1 | 0 |
| 23 | 17 | G | 2 | 0 |
| 24 | 10 | G | 1 | 0 |

(M = Male, F = Female, G = Gelding)

Intravenous administration of the cells was effective in producing improvement on the lameness scale in all horses with osteoarthritis and this response was following a single dose of MSC (Table 1); however, there was some variability in the response.

Long-term culture of MSC can affect their phenotype, inducing changes in cell morphology accompanied by a change in cytokine secretion, reduced adhesion, slower doubling rates and a shift in potency towards adipogenesis. For these reasons, the cells used in this study were all from early passages (e.g., passage 3-5). Nevertheless, it is possible that the animals' age, general health or medications could also affect their response to the infusions.

MSCs actively suppress T cell proliferation in a dose dependent manner by production of anti-inflammatory cytokines. Analysis of cytokines demonstrated up-regulation of IFN-gamma and IL-10, and down-regulation of TNF-alpha production relative to control cultures.

When administered IV, MSCs through a complex array of paracrine-derived activities exert systemic anti-inflammatory effects including a decrease in splenic and cardiac NK cells. This is of interest because NK cells are key regulators of both the innate and adaptive immune responses. MSCs secrete numerous growth factors and cytokines influencing a diverse array of pathways, such as those related to multiple inflammatory pathways, angiogenesis, tissue healing, apoptosis, mitochondrial dysfunction, microvascular dysfunction, and collagen deposition.

In order to enhance safety and efficacy of this cell therapy, the CliniMACS Prodigy will be used to achieve GMP-compliant cell separation and cell culture. The equipment will be programmed to perform all the procedures described below (Study Design—EA-ESC Isolation and Culture), from the moment the blood arrives to the laboratory at University of Alabama until the cells are plated in flasks.

ClinicalMACS Prodigy is equipped with a gas-mix unit for controlled feed of CO2, N2, and air into the CentriCult Unit. This Unit has an active temperature control and permits centrifugations, temperature-controlled incubations that reduces hands-on time and individual process variability.

In-process control of cell density will be achieved via an integrated cell culture microscope and media exchange as well as addition of supplements and growth factors and the final cell harvest will be programmed into a fully automated cell culture process.

### Example 8. General Methodology

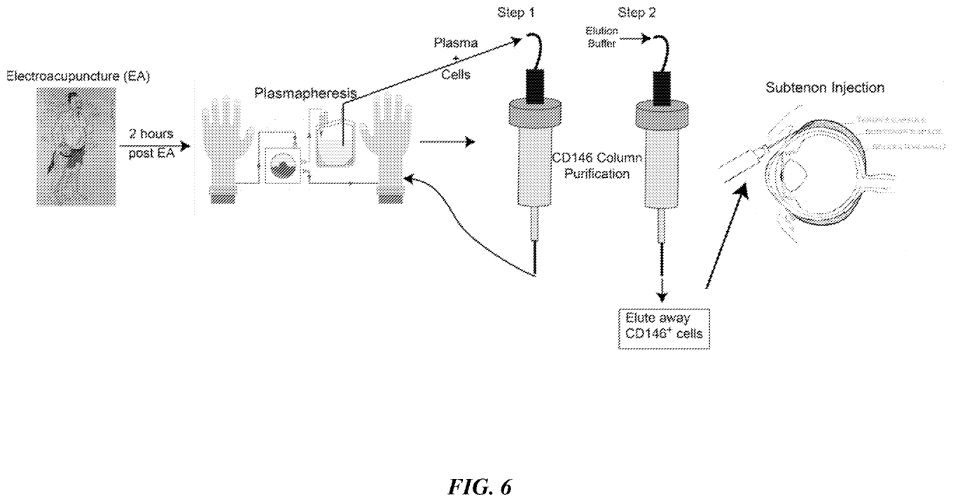

A subject having age-related macular degeneration undergoes an approximately 45 minute electroacupuncture (EA) session during which the LI-4, LI-11, GV-14 and/or GV-20 acupoints are stimulated. Approximately 2 hours following the EA session, the subject undergoing leukaphoresis to separate white blood cells from red bloods cells, and the red blood cells are returned to the subject. The white blood cells are then purified using a column that contains anti-CD146 antibodies. The CD146⁺ cells bound to the antibodies are eluted from the column, concentrated (e.g., by centrifugation), reconstituted in sterile saline, and administered to the sub-Tenon's space of the subjects eye. The procedure is optionally repeated at 3- to 6-month intervals. See, e.g., FIG. 6.

### Example 9. Clinical Study

The objective of this study is to evaluate the efficacy of autologous EA-mobilized and culture expanded MSC for improving lameness and ultrasound appearance of naturally occurring acute superficial digital flexor tendon (SDFT) core lesions (tendonopathy). Both subjective and objective measures of lameness and ultrasound changes will be used to make this determination.

Animal Subjects

Adult horses within a defined competitive athletic age group (2 to 15 years of age) of mixed sex with a recent (≤14 days) core lesion of a forelimb SDFT. Horses should present for initial study evaluation no sooner than 7 days after the injury (first appearance of swelling and lameness) and no later than 14 days after the injury. The delay in initial evaluation is because core lesions can expand for the first seven days after injury and the initial ultrasound should reflect the core lesion at its maximal size. Horses are permitted to have received oral, intravenous, and/or topical non-steroidal anti-inflammatories drugs (NSAIDs), cold therapy, and bandaging as is customary for these lesions, prior to enrollment. However, the horse must not have received any local injections into the tendon, shockwave therapy, laser therapy, tendon splitting, or any other treatment not included in the permitted list.

Inclusion Criteria
Age 2-15 years
Acute core lesion of a forelimb SDFT ≤14 days from the time of injury
Must be a first occurrence of SDFT injury in the affected limb
Diagnosis on ultrasound at UF Large Animal Hospital or by regular attending veterinarian
Prior treatment with oral, intravenous, and/or topical NSAIDs, cold therapy, and bandaging are the only treatments permitted prior to study enrollment
Horses must not have received NSAIDs for 48 hours prior to electroacupuncture
Horse will be housed at their farms but will go to University of Florida Equine Clinic e in Reddick Florida for initial evaluation and for subsequent evaluations as per schematic of study design.
Completion of consent form by the owner or designated owner representative

Exclusion Criteria
Age <2 or >15 years
Generalized tendinopathy without a core lesion
Re-injury of the same limb
Significant confounding lameness/musculoskeletal disease
Systemic disease
Tendon lesion >14 days from the time of injury
Administration of any treatment other than those allowed for in the inclusion criteria
Treatment with any therapy other than those described in the study protocol

Randomization

Upon enrollment, horses will be randomly assigned to either the treatment or control group. Sealed numbered envelopes (1-60) containing a card with the group assignment printed clearly (30—EA-MSC and 30—control) will be mixed and kept in a box. The envelopes will have been prepared and envelope number and corresponding treatment documented by the stem cell laboratory technician. A random envelope will be withdrawn by clinical study staff and the number of the envelope recorded as the Treatment Number. The sealed envelope will be mailed with blood collected for stem cell isolation and expansion and the number and treatment assignment verified by the stem cell laboratory technician. The stem cell laboratory technician preparing the injection will be the only study participant privy to the study group designation of each horse and will maintain a spreadsheet recording this information. The stem cell laboratory technician will be responsible for cell culture and providing the appropriate injection but will have no role in clinical evaluation of horses.

Signalment and History Collection

Each horse's medical record number (MRN), owner information, signalment (breed, age, and sex), use or intended use, date of injury, treatments administered, and any pertinent historical medical information will be recorded.

Subjective and Objective Lameness Exam

A general physical exam, passive musculoskeletal exam, active lameness exam, and lameness locator results will be recorded at study entry (baseline), and 1, 3, and 6 months after study entry. Subjective assessments of pain response on palpation of the lesion, tendon softening, and degree of bowing of the tendon will be recorded for all passive exams. Horses will be walked on hard and soft ground in hand on a straight line. Horses will be instrumented with sensors and trotted in hand on hard ground in a straight line for 3 full passes. Lameness will be graded subjectively according to AAEP lameness grades. Only after determination of the AAEP lameness scale will the objective lameness locator data be revealed and recorded. Horses determined to be a significant risk of injury if trotted will not be trotted and the reason recorded.

Ultrasound Evaluation

Ultrasound evaluation will be performed at study entry (baseline), and 1, 3, and 6 months after study entry. The horse will be sedated with xylazine, and the palmar metacarpus of the affected limb clipped and cleaned. Coupling gel will be applied to the limb. Ultrasound will be performed with a 7-15 MHz linear probe using an echoleucent standoff pad in longitudinal and transverse planes. Prior to starting the study, machine settings for depth, focal position, gain, and probe frequency will be established and the same settings used throughout the study. The distance of the proximal extent, distal extent, and the maximal injury zone from the most prominent point of the accessory carpal bone will be recorded. Transverse images will be obtained every 2 cm from the most proximal to the most distal extent of the lesion and scored for echogenicity on a scale of 1-5 per Marr C M et al. 1993.[35] Longitudinal images will be obtained every 4 cm and linear fiber pattern scored on a scale of 0-5 per Man C M et al. 1993.[35] Measures of cross sectional area of the tendon and cross sectional area of the lesion at the maximal injury zone will be measured in triplicate and an average area calculated. The cross sectional area of the contralateral SDFT at the same distance from the accessory carpal bone will also be measured in triplicate and an average area calculated. The cross sectional area of the lesion as a percent of total tendon cross sectional area and the cross sectional area of the injured tendon compared to the contralateral limb at the location of the maximal injury zone will also be calculated.[36] Owners may elect to have interim ultrasound exams performed at UF or by their regular attending veterinarian, however the costs of any additional exams will not be covered by the study.

Electroaccupuncture and Blood Collection

Electroacupuncture and blood collection will be performed prior to sedation for ultrasound so mobilization is not affected. Electroaccupuncture will be performed using a fine needle (0.30 mm×75 mm, Suzhou Medical Instrument Factory, Jiangsu, China) inserted into each acupoint. The immune points will be targeted; Bai-hui (dorsal midline at the lumbosacral space), GV-14 (cervicothoracic vertebral space C7-T1), left and right LI-4 (depression distal and medial to the base of the second metacarpal bone between the suspensory ligament and medial splint bone), left and right LI-11 (depression cranial to the elbow in the transverse cubital crease which is the crease formed when the elbow is flexed). Each set will be stimulated by electricity with 20 Hz continuous wave and up to a maximum of 3-4 mAMPs for 45 minutes using the EA Instrument (JM-2A model, Wuxi Jiajian Medical Instrument, Inc., Wuxi, China). The output of EA stimulation is gradually increased over the first 15 minutes until the first sign of mild motion of the local tissues is observed. Signs of discomfort are not expected but if observed the intensity will be decreased, or the needle will be removed if reduction in intensity does not eliminate signs. Signs of discomfort would include pawing, head shaking, refusal to stand still, attempting to bite at or rub needles. At 2 hours after EA, the skin overlying the jugular vein will be aseptically prepared and 30 ml of blood will be collected into EDTA from the jugular vein using a 20-g needle. An additional 400 ml of blood will be collected in Serum collect tubes to harvest serum for cell expansion. All blood will be labelled with the horse's name, date of collection, MRN, and treatment ID number.

Horse Serum Preparation

The blood will be collect in 40 Plastic Serum Tubes (Vacutainer, BD). After collection of the whole blood, the tubes will be shipped to Dr. Grant's Lab overnight at Room Temperature. The tubes will be centrifuge at 2,000×g for 10 minutes in a refrigerated centrifuge. The resulting supernatant is designated serum and will be transfer into 50 mL sterile polypropylene tubes. The tubes will be placed into a 56° C. water bath for 25 minutes and will be swirled every 3-5 minutes to ensure uniform heating of the serum. After 25 minutes, the tubes will be removed and cooled slowly to room temperature. After 1 hour the serum will be kept at 4° C. for one hour. The serum will be filtered in 0.22 μm to remove precipitate and then aliquotted in 15 mL tubes. The serum will be stored at −20° C. and the small aliquots will be thawed individually as needed.

EA-ESC Isolation and Culture

Blood will be shipped overnight at temperature room to Dr. Maria Grant's lab at the University of Alabama for processing using the Prodigy system. Mononuclear cells will be isolated by gradient centrifugation. Briefly, blood will be diluted with 1 volume PBS supplemented with 2% FBS, and layered on one volume of Ficoll-Paque (GE Healthcare Bio-sciences, Pittsburgh, Pa.) in 50 mL conical tubes. After 30 min centrifugation at ˜900×g, accel 5, brake 1, room temperature, the buffy coat containing mononuclear cells are collected, and transferred to a new tube, filled with 2% FBS in PBS, supplemented with 1 mM EDTA, and centrifuged 10 min, ˜400×g, accel 9, brake 5, room temperature. A red blood cell lysis step is done by adding over the pellet 5 mL of ammonium chloride solution (Stem Cell Technologies, Vancouver, Canada) and incubating the mixture for 15 minutes at 4° C. Cells were further washed two more times as described.

For in vitro expansion, 4×10⁷ PBMCs will be plated on 150 mm dish in 15 mL of Regular Medium (1:1 dilution of aMEM (Lonza, Walkersville, Md., USA) and EBM-2 (Lonza), with 15% FBS and 1% antibiotics) and place in the incubator 37° C. 5.0% of CO₂. After 2 hours the non-adherent cells will be aspirated and 15 mL of Regular medium will be added. Also the remaining PBMCs will be plated in grade plastic 6-well plates at 10⁷ cells/well in 3 mL. Cells are allowed to attach for 3 days and medium is changed every other day. Colonies usually appeared 10-21 days after plating, and reached 70-80% confluence at 21-30 days post-plating.

Cells will be passaged when cultures reached 80% confluence by detaching with Triple Express (Thermo Scientific), washed twice in PBS+1% of Autologous Horse Serum, as described above. Cells will be expanded in 3 to 5 passages until a minimum of 5×10⁷ cells are obtained, for the time of expansion, in order to obtain cells free of any foreign antigen, the cells will be cultivated in Horse Recombinant Medium (prepared as described below). The cells will be prepared for freezing by resuspending at 5×10⁶ cells/mL in a mixture of 90% Autologous Horse Serum and 10% dimethyl sulfoxide (DMSO, Sigma Aldrich, Carlsbad, Calif.) and slowly cooling to −80° C. Once frozen, they will then be moved to liquid nitrogen for cryopreservation.

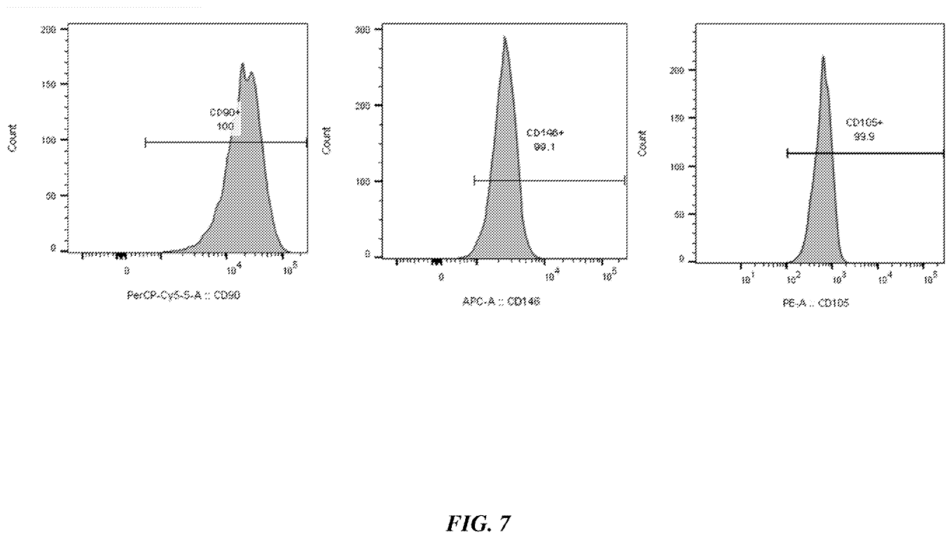

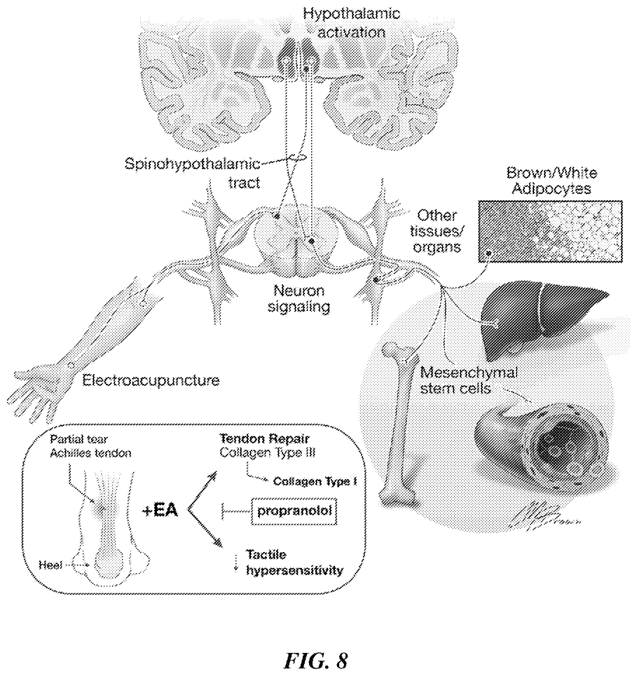

An aliquot of the sample will be kept in the lab for immediate contamination testing for bacteria and fungus, under manufacturing instructions (please see the data sheet of cell culture contamination detection kit from Thermo Fisher, see appendix section), the same sample will be stained with Trypan blue to measure viability. The cells will be stained with antibodies against CD90 (anti-human), CD146 (anti-horse) and CD105 (anti-horse) and analyzed by flow cytometry in order to confirm mesenchymal phenotype. As there is no antibody anti-CD90 horse specific available, we tested human-specific antibodies to stain Horse Mesenchymal cells, results represented in FIG. 7 show that this antibody cross-react with horse's cells and can be used as positive markers to Mesenchymal cells.

Horse Recombinant Medium Preparation

To prepare 100 mL of Horse recombinant medium, which will be a specific medium for each different horse, we will add 42 mL of enriched EBM medium+42 mL of aMEM (Lonza)+15 ml of Autologous Horse serum+1 mL of antibiotics (50 U/ml penicillin-streptomycin (Sigma-Aldrich Japan, Tokyo, Japan).

The enriched EBM medium consist of a mix of horse recombinant factors, as described: for each 100 mL of EBM-2 (Lonza) add 8 μL of IGF (Kingfisher Biotech, Inc) in a concentration of 250 μg/mL+4 μl of FGF Equine (R&D Systems) in a concentration of 250 μg/mL+2 μl of VEGF (Innovative Research) in a concentration of 25 μg/mL+1125 μl of 2 mg/mL Heparin (Stem Cell)+10 μL of 10 mg/mL Ascorbic acid (Sigma)+100 μL of 200 μg/mL Hydrocortisone (Sigma). All the reagents used have the Certificated of Analysis on appendix section.

Intravenous Injection of EA-ESC or Control

Ten cryovials of cells will be shipped in dry ice in a concentration of 5 million of cells/vial. Each vial of 1 mL of cells will be defrost quickly at temperature room and the cells will be diluted in 9 mL of PBS, the total amount of injection will be 50 mL. The injected starting approximately 1 month after the cells were collected, and then every 3 weeks for a total of 3 treatments. While MSC grow rapidly the time required to generate 150 million cells for the three injections will be variable depending on the horse. We will utilize the first 50 million for the first injection and continue to expand the cells until we reach the required additional 100 million cells. Based on our experience, we should be able to achieve this within the second month from the initial culturing of PBMC.

Horses in the control group will have 100 ml of calcium and magnesium phosphate buffered saline drawn up and labelled as above. Only the laboratory technician will have knowledge of the contents of the injection (EA-MSC or Control). The assigned treatment will be shipped in dry ice overnight to the regular attending veterinarian for administration. A “Study Treatment Form” will accompany the vials and will include identifying information as listed on the syringes, injection number (1, 2, or 3), instructions for injection (including the need to roll the syringe between hands for 3 minutes to resuspend the cells prior to injection), and adverse reactions comment section. The skin overlying the jugular vein will be aseptically prepared. A 14 gauge, 5″ catheter will be inserted into the jugular vein and the solution (either EA-MSC or control) injected over 3-5 minutes. Heart rate and respiratory rate will be monitored before and at 5 minute intervals for 15 minutes after the injection is complete. Any adverse events will be recorded.

Rest and Rehabilitation Program

The owner or trainer will be provided with a standardized rest and rehabilitation program in calendar form. Any deviations from the program, adverse events, or need for treatment of a concurrent illness or injury will be recorded. Significant or unjustified deviations from the rest and rehab program or treatment for a concurrent condition (i.e., colic during the study period) will be evaluated on a case-by-case basis and may result in removal of the horse from the study. Any treatment directed at the tendon not explicitly prescribed as part of the study protocol will result in removal of the horse from the study. Stem cells will not be maintained for horses removed from the study except under exceptional circumstances. The rest and rehabilitation program will consist of 2 weeks of strict stall rest following the injury. NSAIDs will be administered for 10 days after injury. The limb will be maintained in a supportive bandage that will be reset daily. The horse will undergo a controlled exercise program outlined on the calendar provided.

Study Exit

Owners will be informed of the horse's treatment or control status only after all data has been collected. The stem cells will be maintained in cryogenic storage for 1 year for all horses completing the study. Cells will not be maintained for future use for horses not completing the study unless due to exceptional circumstances. After 1 year of storage, owners will responsible for the costs of maintaining cryogenic storage. Owners of horses in the control group may elect to have their horse's cells injected after the conclusion of the trial.

Statistical Methods

The following parameters will be assessed using t-tests (or non-parametric equivalent for categorical data) for homogeneity of the treatment and control groups: Age, sex, use, baseline AAEP lameness grade, baseline lameness locator data, baseline ultrasound parameters (ie. lesion length, cross sectional area at maximal injury zone). A repeated measures ANOVA will be used to evaluate lameness grade, lameness locator data, ultrasound parameters (lesion length, cross sectional area at maximal injury zone, echogenicity, linear fiber pattern) of treated compared to control horses.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method, comprising:
(a) electrostimulating at least one acupoint in a subject;
(b) isolating from a blood sample obtained from the subject stem cells that are immunocytochemically positive for CD146 (CD146⁺); and
(c) administering to the subject a pharmaceutical composition of cells comprising non-cultured CD146⁺ stem cells of step (b).

2. The method of claim 1, wherein step (a) comprises electrostimulating at least two acupoints in the subject.

3. The method of claim 1, wherein the at least one acupoint is selected from the group consisting of LI-4, LI-11, GV-14, GV-20, ST-36 and LV-3.

4. The method of claim 1, wherein step (a) comprises applying a pulsating 15-25 hertz (Hz) electric current to acupuncture needle(s) inserted in the at least one acupoint in the subject.

5.-7. (canceled)

8. The method of claim 1, wherein step (b) comprises subjecting the subject to leukaphoresis to separate white blood cells from red blood cells.

9. The method of claim 8 further comprising passing the white blood cells through purification column containing anti-CD146 antibodies.

10. The method of claim 9 further comprising eluting CD146⁺ stem cells bound to the anti-CD146 antibodies in elution buffer.

11. The method of claim 10 further comprising washing the eluted CD146⁺ stem cells to remove the elution buffer.

12. The method of claim 11 further comprising concentrating the CD146⁺ stem cells in sterile saline to produce the pharmaceutical composition of CD146⁺ stem cells.

13. The method of claim 12, wherein the pharmaceutical composition comprises 10,000 to 100,000 CD146⁺ stem cells.

14. The method of claim 12, wherein at least 90% of the cells of the pharmaceutical composition are CD146⁺ stem cells.

15.-22. (canceled)

23. The method of claim 1, wherein the subject has an eye disease.

24. The method of claim 23, wherein the eye disease is selected from macular degeneration, diabetic macular ischemia, and diabetic retinopathy.

25.-29. (canceled)

30. The method of claim 29, wherein the at least two acupoints are selected from the group consisting of LI-4, LI-11, GV-14, GV-20, ST-36 and LV-3.

31. (canceled)

32. The method of claim 31 further comprising cryopreserving non-cultured CD146⁺ stem cells.

33. The method of claim 32 further comprising thawing the cryopreserved non-cultured CD146⁺ stem cells, and administering the non-cultured CD146⁺ stem cells to the subject.

34. (canceled)

35. (canceled)

36. A method, comprising administering to a subject having an eye disease a pharmaceutical composition comprising non-cultured cells that are immunocytochemically positive for CD146 (CD146⁺), wherein the non-cultured CD146⁺ stem cells are isolated from a subject subjected to electroacupuncture, wherein the preparation optionally comprises a pharmaceutically acceptable carrier and/or excipient.

37. The method of claim 36, wherein the eye disease is selected from macular degeneration, diabetic macular ischemia, and diabetic retinopathy

38. The method of claim 37, wherein the eye disease is macular degeneration.

39. A pharmaceutical composition comprising non-cultured cells that are immunocytochemically positive for CD146 (CD146⁺), wherein the non-cultured CD146⁺ stem cells are isolated from a subject subjected to electroacupuncture, wherein the preparation optionally comprises a pharmaceutically acceptable carrier and/or excipient.

40. (canceled)
